(19) United States
(12) Patent Application Publication
(10) Pub. No.: US 2015/0224202 A1
(43) Pub. Date: Aug. 13, 2015

(54) FORMULATIONS AND USES FOR MICROPARTICLE DELIVERY OF ZINC PROTOPORPHYRINS

(71) Applicant: The Board of Trustees of the Leland Stanford Junior University, Palo Alto, CA (US)

(72) Inventors: David K. Stevenson, Palo Alto, CA (US); Jayakumar Rajadas, Cupertino, CA (US); Cecilia Espadas, Stanford, CA (US); Ronald J. Wong, Stanford, CA (US); David Lechuga, Stanford, CA (US)

(21) Appl. No.: 14/612,142

(22) Filed: Feb. 2, 2015

Related U.S. Application Data
(60) Provisional application No. 61/935,200, filed on Feb. 3, 2014.

(51) Int. Cl. A61K 47/44; A61K 47/34; A61K 47/24; A61K 31/555; A61K 47/36

(52) U.S. Cl. CPC A61K 47/44; A61K 31/555; A61K 47/36; A61K 47/24; A61K 47/34

(57) ABSTRACT

Formulations and methods of use thereof that relate to biocompatible delivery of zinc protoporphyrin (ZnPP) are provided. In some embodiments, the ZnPP is formulated for oral delivery. Formulations may include microparticles of ZnPP, wherein the ZnPP active agent is admixed or coated with a pharmaceutically acceptable stabilizer providing for increased stability to acid conditions and improved solubility at neutral pH.

### RELATED APPLICATIONS

This application claims the benefit of priority to U.S. Provisional Patent Application Ser. No. 61/935,200, filed Feb. 3, 2014, which is hereby incorporated by reference herein in its entirety.

### BACKGROUND OF THE INVENTION

Metalloporphyrins are structural analogs of heme and their potential use in the management of neonatal hyperbilirubinemia and other conditions has been the subject of considerable research for more than three decades. The pharmacological basis for using this class of compounds to control bilirubin levels is the targeted blockade of bilirubin production through the competitive inhibition of heme oxygenase (HO), the rate-limiting enzyme in the bilirubin production pathway. Ongoing research continues in the pursuit of identifying metalloporphyrins that are safe and effective, by defining therapeutic windows and targeted interventions for the treatment of excessive neonatal hyperbilirubinemia.

HO enzymes exist as constitutive (HO-2) and inducible (HO-1) isoforms. The heme oxygenases are metabolic enzymes that utilize NADPH and oxygen to break apart the heme moiety liberating biliverdin, carbon monoxide (CO) and iron. Biliverdin (BV) and bilirubin, the substrate and product of biliverdin reductase, respectively, are potent antioxidants. The function of HO-1 in cell homeostasis includes the features of acting as a fundamental ‘sensor’ of cellular stress and direct contributor to limit or prevent tissue damage; participation of the products of HO activity in cellular adaptation to stress. Pharmacological manipulation of the HO-1 pathway and its products can be used for conferring protection against a variety of conditions characterized by oxidative stress and inflammation.

Zinc protoporphyrin (ZnPP) is a normal metabolite that is formed in trace amounts during heme biosynthesis. The final reaction in the biosynthetic pathway of heme is the chelation of iron with protoporphyrin. During periods of iron insufficiency or impaired iron utilization, zinc becomes an alternative metal substrate for ferrochelatase, leading to increased ZnPP formation. Evidence suggests that this metal substitution is one of the first biochemical responses to iron depletion, causing increased ZnPP to appear in circulating erythrocytes. Because this zinc-for-iron substitution occurs predominantly within the bone marrow, the ZnPP/heme ratio in erythrocytes reflects iron status in the bone marrow. In addition, ZnPP may regulate heme catabolism through competitive inhibition of HO, the rate-limiting enzyme in the heme degradation pathway that produces bilirubin and CO. Clinically, ZnPP quantification is valuable as a sensitive and specific tool for evaluating iron nutrition and metabolism. Diagnostic determinations are applicable in a variety of clinical settings, including pediatrics, obstetrics, and blood banking.

ZnPP has some desirable characteristics for treatment of neonatal jaundice and other conditions: it is highly potent; it does not cross the blood-brain barrier (BBB); it is relatively inert to light activation and thus has no photosensitizing/phototoxic effects in vivo; and it is not degraded by HO. However, delivery of the compound has been difficult. The present invention addresses this issue.

### SUMMARY OF THE INVENTION

The invention provides formulations and methods of use thereof that relate to biocompatible delivery of an effective dose of zinc protoporphyrin (ZnPP). In some embodiments, the ZnPP is formulated for oral delivery. These formulations provide microparticles of ZnPP, wherein the ZnPP active agent is coated with a pharmaceutically acceptable excipient. In some embodiments, a therapeutic composition is provided, comprising a coated microparticle comprising ZnPP, and a pharmaceutically acceptable excipient. The therapeutic composition may be formulated for oral administration, including without limitation for administration to neonates and infants, e.g., as a liquid, through a gastric tube, etc.

The microparticles described herein comprise the ZnPP active agent and a pharmaceutically acceptable stabilizer, e.g., where the active agent may be at least about 5% of the total microparticle weight, and preferably not more than about 50% of the total microparticle weight. The stabilizer can protect the active agent from instability at low pH, e.g., the acidic conditions present in the stomach. The stabilizer may also increase the solubility of the active agent in neutral pH, e.g., to increase absorption in the neutral conditions present in the intestine.

The microparticles may be suspended in a pharmaceutically acceptable carrier to provide a sufficiently concentrated formulation to deliver the desired dose of active agent in a reasonable volume of the formulation. Carriers include pharmaceutically acceptable excipients, including aqueous excipients. Such compositions can be provided in a unit dose formulation, e.g., comprising a dose of microparticle ZnPP for administration to a patient. The unit dose will also typically further comprise excipients, e.g., excipients that provide for enhanced stability and solubility. In certain embodiments, an effective dose of active agent is that dose which, when provided to a patient, is effective in inhibiting inducible heme oxygenase (HO-1), but to a greater degree than it inhibits constitutive heme oxygenase (HO-2), preferably without substantially inhibiting constitutive heme oxygenase (HO-2). In certain embodiments, an effective dose stimulates increased degradation of bilirubin in an infant or neonate, relative to a control in the absence of treatment with the compositions or methods described herein.

Methods of treatment with formulations and compositions described herein are also provided. In some embodiments, a method is provided for treating hyperbilirubinemia with ZnPP, comprising the steps of administering an effective dose of a microparticulate formulation of ZnPP described herein to an individual in need thereof.

More particularly, embodiments disclosed include methods of treating hyperbilirubinemia or the symptoms thereof in an infant. In some embodiments, the infant is of a gestational age from about 35 to about 43 weeks. In other embodiments the infant is not more than 30 days of age. In some embodiments, the infant has a minimum birth weight of about 2,500 g. In some embodiments, the infant has a birth weight from about 1,700 g to about 4,000 g. In some embodiments, the infant has at least one risk factor, e.g., hemolytic disease, ABO blood type incompatibility, anti-C Rh incompatibility, anti-c Rh incompatibility, anti-D Rh incompatibility, anti-E Rh incompatibility, anti-e Rh incompatibility, glucose-6-phosphate dehydrogenase (G6PD) deficiency, or any combination thereof. In some embodiments, a ZnPP formulation described herein is administered at a time selected from within about 6 hours of birth, within about 12 hours of birth, within about 24 hours of birth, and within about 48 hours of birth. In some embodiments, the ZnPP formulation is orally administered.

Some embodiments further comprise determining post treatment total bilirubin levels following administration of the ZnPP formulation. In some embodiments, post treatment total bilirubin levels are at least 5% below the baseline total bilirubin (TB) levels 24 hours after administering a therapeutic amount of a ZnPP formulation to the infant. In some embodiments, post treatment TB levels are at least 10% below the baseline TB levels 48 hours after administering a therapeutic amount of a ZnPP formulation to the infant. In some embodiments, post treatment TB levels are at least 20% below the baseline TB levels 72 hours after administering a therapeutic amount of a ZnPP formulation to the infant. In some embodiments, post-treatment TB levels are less than 3 mg/dL above the baseline TB levels 48 hours after administering a therapeutic amount of a ZnPP formulation to the infant.

The formulations described herein also find use in other methods where the delivery of an effective dose of ZnPP is desired. In some embodiments, methods of treating cancer are provided. HO-1 has been shown to be involved in pro-tumoral activities. By inhibiting HO, ZnPP formulations described herein can exert anti-tumor activity, alone or in combination with chemotherapy or radiotherapy, e.g., in the treatment of solid tumors such as carcinomas, etc.

Other methods include, without limitation, treatment of age-related macular degeneration, treatment of infection, etc., in various conditions where selective inhibition of HO is desired. The compositions described herein also find use as contrast enhancing agents for NMR imaging.

### BRIEF DESCRIPTION OF THE DRAWINGS

FIG. 1 provides a schematic representation of the heme degradative pathway. Reduction of bilirubin production can be targeted through the inhibition of heme oxygenase (HO), the rate-limiting enzyme in the heme degradative pathway. Heme is degraded by HO to produce equimolar quantities of carbon monoxide (CO) and biliverdin, which is then immediately degraded by biliverdin reductase to form bilirubin.

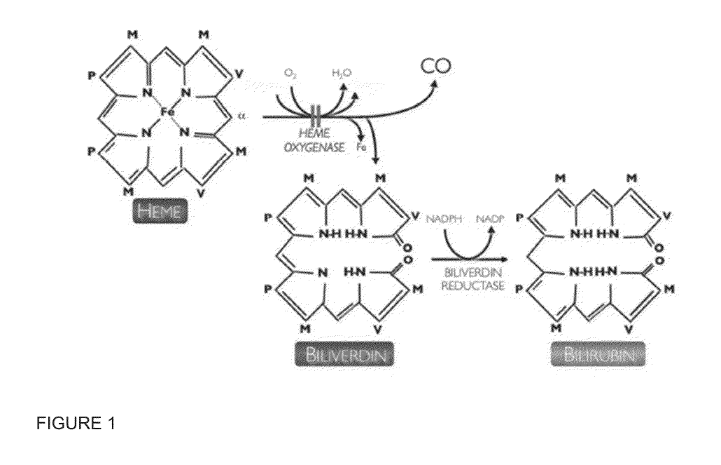

FIG. 2 includes three panels ((A), (B), and (C)), showing the experimental setup for phototoxicity studies. 3-day-old pups (magnified in panel (A)) were given vehicle or Mp IP at doses ranging from 3.75 to 30 μmol/kg body weight (BW) and then exposed for 3 hours to fluorescent light consisting of 2 cool white and 1 blue tubes (as shown in panel (B)) emitting an irradiance of 35.0±1.0 μW/cm²/nm as measured by a BiliBlanket II Meter (as depicted in panel (C)).

FIG. 3 depicts data summarizing the safety and efficacy of ZnBG. Administration of 3.75 μmol ZnBG/kg BW to 3-day-old pups exposed to light showed no phototoxicity as shown by a survival of 100% (empty bar) and inhibited HO activity (filled bar) up to 75%.

FIG. 4 depicts in vitro inhibition of HO activity after contact with various zinc formulations. The ZnPP formulations at 30 μM were evaluated for in vitro HO inhibitory potency using liver, spleen, and brain sonicates harvested from 3-day-old mouse pups.

FIG. 5 includes two panels showing intragastric injections of ZnPP formulations. ZnPP formulations at a dose of 30 μmol/kg BW were administered to 3-day-old mouse pups via direct intragastric (IG) injections. FIG. 5 also includes four panels showing the gastric contents 3 hours after administration of formulations: aqueous ZnPP phosphate (ZnPP-PO₄); ZnPP-chitosan (ZnPP-A and ZnPP-B); ZnPP EUDRAGIT® (ZnPP-Poly); and ZnPP phospholipids (Zn PP Lipid).

FIG. 6 (A) depicts In vivo inhibition of HO activity. Formulations or VEH were injected IG into 3-day-old newborn FVB mouse pups. 3 hours after administration, liver, spleen, and brain were harvested and HO activity measured by gas chromatography (GC). Inhibition was expressed as % of control values.

FIG. 7 depicts two panels ((A) and (B)) showing SEM images for spray-dried particles with: (A) 75% w/w EUDRAGIT®; and (B) 38% EUDRAGIT®. Wrinkled morphology indicates early polymer precipitation during particle formation ensuring efficient drug encapsulation.

FIG. 8 depicts two panels ((A) and (B)). Panel (A) shows the stomach contents of a pup administered ZnPP-PO₄ in sodium phosphate buffered solution; the protoporphyrin precipitated in the stomach. Panel (B) shows the stomach contents of a pup administered ZnPP spray-dried microparticles; the spray-dried microparticles do not precipitate.

### DETAILED DESCRIPTION OF THE EMBODIMENTS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a microsphere” includes a plurality of such microspheres and reference to “the stent” includes reference to one or more stents and equivalents thereof known to those skilled in the art, and so forth.

The terms “treating”, and “treatment” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a condition, symptom or adverse effect attributed to the condition. The term “treatment” as used herein covers particularly the application of a composition comprising an ZnPP active agent in microparticle form, including oral administration. The term “prophylaxis” is used herein to refer to a measure or measures taken for the prevention or partial prevention of a disease or condition.

The term “prevent” is art-recognized, and when used in relation to a condition is well understood in the art, and includes administration of a composition which reduces the likelihood of, or delays the onset of, the condition in a subject relative to a subject which does not receive the composition. Thus, prevention of hyperbilirubinemia includes, for example, reducing the likelihood that a subject receiving the composition will experience hyperbilirubinemia relative to a subject that does not receive the composition, and/or delaying the onset of hyperbilirubinemia, on average, in a treated population versus an untreated control population, e.g., by a statistically and/or clinically significant amount.

The term “subject” includes mammals, e.g., cats, dogs, horses, pigs, cows, sheep, rodents, rabbits, squirrels, bears, and primates such as chimpanzees, gorillas, and humans.

Physiologic jaundice is common during the transitional period (1 week after birth) and is observed in 60% to 70% of term infants. The condition results from the abrupt cessation of bilirubin clearance by the placenta and transient deficiencies in hepatic bilirubin uptake, intracellular transport, and glucuronosyltransferase (UGT1A1) conjugation activity. A major contributing factor is the 2- to 3-fold increased rate of bilirubin production in neonates as compared to adults. The heme degradation process produces equimolar amounts of CO and bilirubin. Neonates with isoimmune hemolytic disease (e.g., Rhesus and ABO incompatibility) or other hemolytic conditions (e.g., G6PD deficiency) typically have elevated bilirubin production rates in association with increased TB levels. Historically, increased TB concentrations in the context of hemolytic disease have been associated with neurotoxicity and brain injury (kernicterus), and have prompted aggressive and relatively risky interventions, such as exchange transfusion.

The risk for low IQ scores (at adulthood) may be significantly greater in term male infants with severe non-hemolytic jaundice. Also, there has been an increase in the number of reported cases of kernicterus in apparently healthy term and near-term breastfed infants or those with unidentified hemolytic disorders. Moreover, results from a recent randomized controlled trial suggested that aggressive phototherapy significantly reduced the rate of neurodevelopmental impairment (NDI), but may be counterbalanced by an increase in mortality among extremely low birth weight (ELBW) infants (weighing 501 to 750 g), further emphasizing the need for preventive methods, including pharmacological approaches to modulate bilirubin production and thus reduce the likelihood of dangerously high bilirubin levels in this high-risk group.

Currently approved and commonly used treatments for hyperbilirubinemia include phototherapy and exchange transfusion. Phototherapy involves irradiating the newborn with light in the 430 to 490 nm range (blue light). The light converts bilirubin into lumirubin and photobilirubin, which are less toxic water-soluble photoisomers that are more readily excreted by the infant, and thus can result in a reduction of bilirubin levels.

The term “pharmaceutically acceptable” as used herein refers to a compound or combination of compounds that will not impair the physiology of the recipient human or animal to the extent that the viability of the recipient is compromised. Preferably, the administered compound or combination of compounds will elicit, at most, a temporary detrimental effect on the health of the recipient human or animal.

The term “carrier” as used herein refers to any pharmaceutically acceptable excipient, diluent, or other dispersant of agents that will allow a therapeutic composition to be administered by the desired route, e.g., by oral administration. A “carrier” as used herein, therefore, includes such excipients and compounds known to one of skill in the art that are pharmaceutically and physiologically acceptable to the recipient human or animal.

The formulations described herein comprise stabilized microparticles of ZnPP as described above where, relative to the uncoated ZnPP, the microparticles have increased stability in acidic conditions, and/or enhanced solubility at neutral pH. At acidic conditions, e.g. at a pH not more than 4.5, not more than 4.3, not more than 4, not more than 3.5, ZnPP precipitates and degrades to inactive components. In some embodiments, the microparticles are at least 10% more stable to acidic conditions, at least 20% more stable, at least 30% more stable, at least 40% more stable, at least 50% more stable, at least 75% more stable, and may be at least 2-fold more stable, at least 5-fold, at least 10-fold or more. Stability can be experimentally determined by observing precipitation and degradation in experimental conditions in vitro.

Stabilized microparticle formulations of the invention also confer enhanced absorption of ZnPP at neutral pH, e.g., at a pH greater than 5.5 but less than 8.5, including a pH of greater than about 6.0, greater than about 6.5, greater than about 7.0, and less than about 8.5, less than about 8.0. In some embodiments the microparticles are at least 10% more soluble in neutral pH, at least 20% more soluble, at least 30% more soluble, at least 40% more soluble, at least 50% more soluble, or at least 75% more soluble, and may be at least 2-fold more soluble, at least 5 fold, at least 10-fold or more. Solubility can be experimentally determined by conventional methods.

The microparticle can comprise or consist essentially of an active agent and a stabilizer. In some embodiments, the concentration of the active agent in the microparticle is up to about 5%, up to about 10%, up to about 15%, up to about 20%, up to about 25%, up to about 30%, up to about 35%, up to about 40%, up to about 45%, up to about 50% of the total weight, and the like, and may be from about 5% to about 50%, from about 10% to about 40%, from about 15% to about 35%, from about 20% to about 30% by weight, preferably about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight.

The balance of the microparticle weight is typically provided by stabilizer, i.e., up to about 95%, up to about 90%, up to about 85%, up to about 80%, up to about 75%, up to about 70%, up to about 65%, up to about 60%, up to about 55%, up to about 50%, up to about 45%, up to about 40% of the total weight. In some embodiments, the concentration of stabilizer in the microparticle is preferably about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, or about 70% by weight. The stabilizer confers increased stability at acidic conditions, and allows for increased solubility at neutral pH conditions.

The microparticles may have a controlled size, as appropriate for optimization of drug delivery. Usually the particle will have a diameter of up to about 10 nm, up to about 50 nm, up to about 100 nm, up to about 250 nm, up to about 500 nm, up to about 1 μm, up to about 2.5 μm, up to about 5 μm, and not more than about 10 μm in diameter. In some embodiments, the microparticle size is from about 100 nm to about 5 μm in diameter, for example from about 100 to about 500 nm, from about 500 nm to about 1 μm, and the like. A plurality of microparticles optionally has a defined average size range, which may be substantially homogeneous, where the variability may not be more than 100%, 50%, or 10% of the diameter. Diameters of microparticles may be measured, for example, using scanning electron microscopy (SEM).

Microparticles can be formed by various methods, including, in some embodiments, the methods exemplified herein. Methods of interest may include, without limitation, controlled cation-induced micro-emulsion; and spray drying. Polymeric microparticle fabrication methods can involve polyelectrolyte complex formation, double emulsion/solvent evaporation techniques, emulsion polymerization techniques, and the like. Spray drying is a process that uses jets of dissolved or suspended drug in an aqueous or other fluid phase that is forced through high pressure nozzles to produce a fine mist. Often, a bulking agent will be added to the fluid as well. The aqueous or other liquid contents of the mist evaporate, leaving behind a fine powder. A modification of spray drying, called air nebulization spray drying, uses two wedge-shaped nozzles through which compressed air passes and liquid solutions pass at high velocity. The wedge-shaped nozzle acts as a fluid acceleration zone where the four streams collide at high velocity, producing a shock wave that generates fine droplets. The droplets then descend into a column while being dried into a solid powder by heated air before being collected.

Stabilizers of interest include, without limitation, alginate, chitosan, lecithin, which are naturally occurring mixtures of diglycerides of stearic, palmitic, and oleic acids, linked to the choline ester of phosphoric acid; sodium trimetaphosphate; poloxamers, i.e., nonionic triblock copolymers composed of a central hydrophobic chain of polyoxypropylene (poly(propylene oxide)) flanked by two hydrophilic chains of polyoxyethylene (poly(ethylene oxide)), including various sizes, e.g., Poloxamer 188, poloxamer 407, etc.; cationic lipids, particularly phospholipids; oils, such as coconut oil, etc. Chitosan is a linear polysaccharide composed of randomly distributed β-(1,4)D-glucosamine and N-acetyl-D-glucosamine. Other stabilizers of interest include, for example a protein, such as albumin (for example bovine serum albumin, human serum albumin, etc.), and polyvinylpyrrolidone (PVP) (a water-soluble branched polymer of N-vinylpyrrolidone). PVP useful in the compositions and methods described herein may have a molecular weight of about 10K, or higher, e.g., from about 20K to 50K.

The term “cationic lipids” is intended to encompass molecules that are positively charged at physiological pH, and more particularly, constitutively positively charged molecules, comprising, for example, a quaternary ammonium salt moiety. Cationic lipids used in the methods of the invention typically consist of a hydrophilic polar head group and lipophilic aliphatic chains. See, for example, Farhood et al. (1992) Biochim. Biophys. Acta 1111:239-246; Vigneron et al. (1996) Proc. Natl. Acad. Sci. (USA) 93:9682-9686.

Cationic lipids of interest include, for example, imidazolinium derivatives (WO 95/14380), guanidine derivatives (WO 95/14381), phosphatidyl choline derivatives (WO 95/35301), and piperazine derivatives (WO 95/14651). Examples of cationic lipids that may be used in the present invention include 1,2-distearoyl-sn-glycero-3-phosphocholine (DSPC); 1,2-dipalmitoyl-sn-glycero-3-phosphocholine (DPPC); DOTIM (also called BODAI) (Solodin et al., (1995) Biochem. 34: 13537-13544), DDAB (Rose et al., (1991) BioTechniques 10(4):520-525), DOTMA (U.S. Pat. No. 5,550,289), DOTAP (Eibl and Wooley (1979) Biophys. Chem. 10:261-271), DMRIE (Feigner et al., (1994) J. Biol. Chem. 269(4): 2550-2561), EDMPC (commercially available from Avanti Polar Lipids, Alabaster, Ala.), DCChoI (Gau and Huang (1991) Biochem. Biophys. Res. Comm. 179:280-285), DOGS (Behr et al., (1989) Proc. Natl. Acad. Sci. USA, 86:6982-6986), MBOP (also called MeBOP) (WO 95/14651), and those described in WO 97/00241.

In some embodiments, the ZnPP is stabilized in a microparticle formulation with a cationic lipid or lipids. Lipids of interest include any of those listed above, e.g., including DSPC, DPPC, DOTIM, DDAB, DOTMA, DMRIE, EDMPC, DCChoI, DOGS, MBOP, etc., which may be used singly or as a cocktail of different lipids, e.g., two lipids at a 10:1, 5:1, 2:1, 1:1, 1:2, 1:5, 1:10, etc. ratio. The lipids can comprise up to about 90% of the microparticle, up to about 85% of the microparticle, up to about 80% of the microparticle, up to about 75% of the microparticle, up to about 70% of the microparticle, up to about 65% of the microparticle, or up to about 50% of the microparticle by weight, where the balance can be the active agent, or can be combined with, for example, EUDRAGIT® L 30 D-55, which is an aqueous dispersion of anionic polymers with methacrylic acid as a functional group, at a concentration of from about 35% to about 75% of the formulation weight. In some embodiments, however, the microparticles are free of EUDRAGIT®. In certain embodiments, the microparticles comprise about 10% to about 25% ZnPP by weight, and the balance is a mixture of DSPC and DPPC in the ratios described above.

In some embodiments, the ZnPP is stabilized in a microparticle formulation with a mixture comprising lecithin, a poloxamer, a neutral oil carrier, e.g., coconut oil, and one or more of alginate, sodium trimetaphosphate and chitosan. In certain embodiments, the microparticles comprise about 5% to about 25% ZnPP, about 10% to about 20% ZnPP by weight. In some such embodiments, the lecithin is present at a concentration of from about 10% to not more than about 40%, from about 20% to not more than about 30% by weight of the microparticle. In some such embodiments, the poloxamer is present at a concentration of from about 10% to not more than about 40%, from about 15% to not more than about 25% by weight of the microparticle. The balance of the formulation comprises, consists essentially, or consists of the neutral oil carrier and the stabilizer.

In some such embodiments, the stabilizer is alginate, which is present at about 3% to about 6%, at about 4% to about 5%, and may be about 4.5% by weight of the microparticle.

In some such embodiments, the stabilizer is chitosan, which is present at about 3% to about 6%, at about 4% to about 5%, and may be about 4.5% to 5% by weight of the microparticle.

In some such embodiments, the stabilizer is sodium trimetaphosphate, which is present at about 3% to about 6%, at about 4% to about 5%, and may be about 4.5% to 5% by weight of the microparticle.

Preferred pharmaceutical compositions are formulated for oral delivery. In some embodiments the microparticles of the invention are provided, e.g., in a unit dose, such as a dry powder for reconstitution immediately prior to administration. Pharmaceutical compositions can include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, or non-toxic, nontherapeutic, non-immunogenic stabilizers, excipients and the like. The compositions can also include additional substances to approximate physiological conditions, such as pH adjusting and buffering agents, toxicity adjusting agents, wetting agents and detergents.

In other embodiments, an oral delivery formulation is provided as a thin film, for example where a dried powder formulation of microparticles is dispersed in a solvent containing a film forming polymer, which can be cast in a thin film and packaged, for example as a unit dose.

Other oral formulations include, without limitation, tablets, lozenges, capsules, sprinkles, sachets, stick-packs, etc. as known in the art and adapted for the microparticles of the invention.

Based on the above, it will be understood by those skilled in the art that a plurality of different treatments and means of administration can be used to treat a single patient.

The total dose per day is preferably administered at least once per day, but may be divided into two or more doses per day. Some patients may benefit from a period of “loading” the patient with a higher dose or more frequent administration over a period of days or weeks, followed by a reduced or maintenance dose.

Specific information regarding formulations which can be used in connection with aerosolized delivery devices are described within Remington's Pharmaceutical Sciences, A. R. Gennaro editor (latest edition) Mack Publishing Company. For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure. The effective amount of a therapeutic composition to be given to a particular patient will depend on a variety of factors, several of which will be different from patient to patient. A competent clinician will be able to determine an effective amount of a therapeutic agent. The compositions can be administered to the subject in a series of more than one administration.

Those of skill will readily appreciate that dose levels can vary as a function of the specific compound, the severity of the symptoms and the susceptibility of the subject to side effects. Some of the drugs are more potent than others. Preferred dosages for a given agent are readily determinable by those of skill in the art by a variety of means. A preferred means is to measure the physiological potency of a given compound.

### Kits and Packaging

In some embodiments, formulations are provided for use in the methods of the invention. Such formulations may comprise a stabilized microparticle of ZnPP, etc., which can be provided in a packaging suitable for clinical use, including packaging as a lyophilized, sterile powder; packaging of a stable suspension of, for example, microparticles, in carrier; separate packaging of microparticles and carrier suitable for mixing prior to use; and the like. The packaging may be a single unit dose, providing an effective dose of an ZnPP active agent in microparticle form in the manufacture of a medicament for improving patient function suffering from hyperbilirubinemia.

### Treatment Methods

Methods described herein include the administration, preferably oral administration, of a pharmaceutical composition comprising the ZnPP-containing microparticles described herein in a dose effective to inhibit the HO enzyme. Oral administration allows targeted delivery by taking advantage of “first pass effect” resulting in localization to liver and spleen. The ZnPP can also be systemic after oral delivery. The effective dose may vary depending on the age of the individual, the condition being treated, and the like.

Embodiments include methods of treating hyperbilirubinemia or the symptoms thereof in an infant. In some embodiments, the infant is of a gestational age from about 35 to about 43 weeks. In other embodiments, the infant is not more than 30 days of age. In some embodiments, the infant has a minimum BW of about 2,500 g. In some embodiments, the infant has a BW from about 1,700 g to about 4,000 g. In some embodiments, the infant has at least one risk factor, e.g., hemolytic disease, ABO blood type incompatibility, anti-C Rh incompatibility, anti-c Rh incompatibility, anti-D Rh incompatibility, anti-E Rh incompatibility, anti-e Rh incompatibility, G6PD deficiency, and combinations thereof. In some embodiments, administering a therapeutic amount of a ZnPP formulation is performed at a time selected from within about 6 hours of birth, within about 12 hours of birth, within about 24 hours of birth, and within about 48 hours of birth. In some embodiments, the ZnPP formulation is orally administered.

Metalloporphyrins appear to begin having an effect about 6-12 hours after administration. Administering ZnPP as disclosed herein can decrease the incidence of or need for phototherapy or exchange transfusions. In some embodiments, administering a ZnPP as disclosed herein may reduce the duration of phototherapy. In some embodiments, administering ZnPP as disclosed herein may reduce the light intensity of phototherapy. In some embodiments, administering ZnPP as disclosed herein obviates the need for phototherapy.

In therapeutic use as agents for treating hyperbilirubinemia, ZnPP may administered at the initial dosage of about 0.1 mg to about 20 mg ZnPP/kg BW (IM). In certain embodiments, treatment with the metalloporphyrin is a one-time single dose treatment. In some embodiments, ZnPP is administered in a dosage of from about 0.5 to about 6 mg/kg ZnPP/kg (IM). In some embodiments, ZnPP is administered in a dosage of from about 0.5 mg/kg to about 4 mg/kg, from about 0.5 mg/kg to about 2 mg/kg, from about 0.75 mg/kg to about 1.5 mg/kg, from about 1.5 mg/kg to about 4.5 mg/kg or from about 3.0 mg/kg to about 4.5 mg/kg, including about 1.5 mg/kg, about 3.0 mg/kg and about 4.5 mg/kg. The dosages, however, may be varied depending upon the requirements of the patient, the severity of the condition being treated and the compound being employed. Determination of the proper dosage for a particular situation is within the skill of the art. For example, treatment may be initiated with smaller dosages, which are less than the optimum dose of the compound. Thereafter, the dosage may be increased by small increments until the optimum effect under the circumstance is reached.

Some embodiments further comprise determination of eligibility and screening assessments. In some embodiments, determination of eligibility and screening assessments include, but are not limited, to transcutaneous bilirubin (TcB) monitoring, an audiology examination including auditory brainstem response (ABR) (also known as automated auditory brainstem response [A-ABR] or brainstem auditory evoked potential [BAEP]), 12-lead ECGs, review of maternal and subject demographic data, review of subject's medical history, review of inclusion and exclusion factor, review of concomitant medication of subjects, assessment of vital signs, physical examination, including weight, length, head circumference, and eyes, dermatological examination, an Amiel-Tison neurologic examination, blood sampling for the following analyses: clinical chemistry, hematology (including blood smear), pharmacokinetics, and combinations thereof.

Some embodiments further comprise a continued evaluation of the subject before treatment, during treatment, after treatment or a combination thereof. In some embodiments, continued evaluation includes, but is not limited to, cB monitoring, an audiology examination including ABR (also known as A-ABR or BAEP), three 12-lead ECGs, review of maternal and subject demographic data, review of subject's medical history, review of inclusion and exclusion factor, review of concomitant medication of subjects, assessment of vital signs, physical examination, including weight, length, head circumference, and eyes, dermatological examination, an Amiel-Tison neurologic examination, blood sampling for the following analyses: clinical chemistry, hematology (including blood smear), pharmacokinetics, and combinations thereof. In some embodiments, vital signs comprise measuring temperature (axillary), blood pressure (measured with age- and size-appropriate equipment), pulse rate, respiratory rate and combinations thereof.

The formulations described herein also find use in other methods where the delivery of an effective dose of ZnPP is desired. In some embodiments, methods of treating cancer are provided. HO-1 has been shown to be involved in pro-tumoral activities. By inhibiting HO, ZnPP in the formulations described herein can provide for an anti-tumor activity, alone or in combination with chemotherapy or radiotherapy, e.g., in the treatment of solid tumors such as carcinomas, etc.

Other methods include, without limitation, treatment of age-related macular degeneration, treatment of infection, etc., in various conditions where selective inhibition of HO is desired. The compositions described herein also find use as contrast enhancing agents for NMR imaging.

Although the present invention has been described in considerable detail with reference to certain preferred embodiments thereof, other versions are possible. Therefore, the spirit and scope of the appended claims should not be limited to the description and the preferred versions contained within this specification.

### EXAMPLE 1

### Effects of Light on Metalloporphyrin-Treated Newborn Mice

Neonatal hyperbilirubinemia arises from an imbalance between bilirubin production and its elimination. A study on aggressive vs conservative phototherapy with extremely low birthweight (ELBW) infants showed that the rate of neurodevelopmental impairment (NDI) alone was significantly reduced with aggressive phototherapy, but this reduction was offset by an increase in mortality among infants ≦501-750 g. In addition, the photo-oxidizing effect of light, including blue light, has been shown in animals. Moreover, it has also been suggested that the lower hemoglobin levels in ELBW infants may place them at higher risk for phototoxicity because less light is absorbed by hemoglobin in circulation. Metalloporphyrins (Mps) are promising drugs for treating hyperbilirubinemia, but most of them are photosensitizing and subsequently potentially phototoxic. Zinc protoporphyrin (ZnPP) is a promising Mp with sufficient potency, but it has poor insolubility and is not absorbed orally. We have designed and developed ZnPP microparticle formulations using a variety of FDA-approved biodegradable polymers and phospholipids, which improve bioavailability and enhance gastric passage. We found that intragastric administration of ZnPP microparticles at 30 μmol/kg to 3 d-old mice resulted in a 2-fold increase in potency compared to 30-μmol ZnPP/kg in phosphate buffer, without showing signs of phototoxicity. We concluded that using polymeric particulate delivery systems (micro- or nanoparticles) can improve the stability and enhance intestinal absorption of ZnPP, while retaining HO inhibitory potency without photosensitizing effects, and thus is useful in treating neonatal hyperbilirubinemia.

The function of heme oxygenase (HO) in specific biological processes has been studied; evaluating heme turnover in adult and neonatal animal models with the goal of modulating enzyme activity through the administration of metalloporphyrin (Mps) as therapeutic compounds. No particular chemical structural feature of Mps has been uncovered that allows us to predict which compound would be the most effective. Desirable drug properties include a low IC₅₀; lack of photosensitizer activity; oral absorption; should not cross the blood brain barrier; should be short-acting; should not substantially upregulate HO-1 mRNA, protein, or activity; and should not be degraded with the subsequent release of the sequestered metal ion.

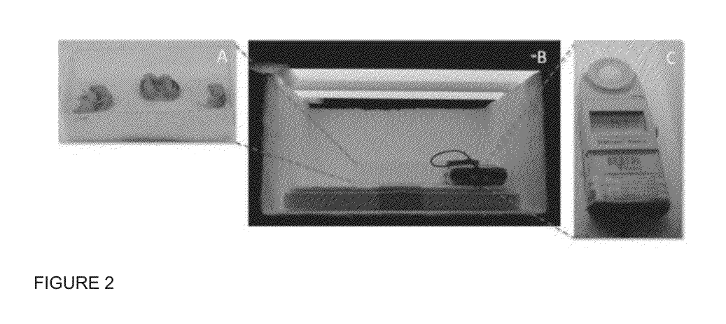

Effect of light exposure on metalloporphyrin-treated newborn mice. We demonstrated the possible photosensitizing effects of two promising Mps for use in the prevention of neonatal jaundice, chromium mesoporphyrin (CrMP) and zinc bis glycol porphyrin (ZnBG). We chose these two compounds because both are orally absorbed, relatively potent with minimal photoreactivity in the desirable dose range, and minimally upregulates HO-1 transcription. In this study, we administered CrMP or ZnBG intraperitoneally (IP) to 3-day-old mouse pups at doses ranging from 3.75 to 30.0 μmol/kg BW. Pups were either kept in the dark (controls) or exposed to fluorescent light composed of two cool white tubes (F20T12cW) and one blue (TL20W/52) tube for 3 hours (FIG. 2). In pups treated with CrMP, we found a dose-dependent mortality with a LD₅₀ (50% lethal dose) of 21.5 and 23.0 μmol/kg for those kept in the dark and exposed to light, respectively.

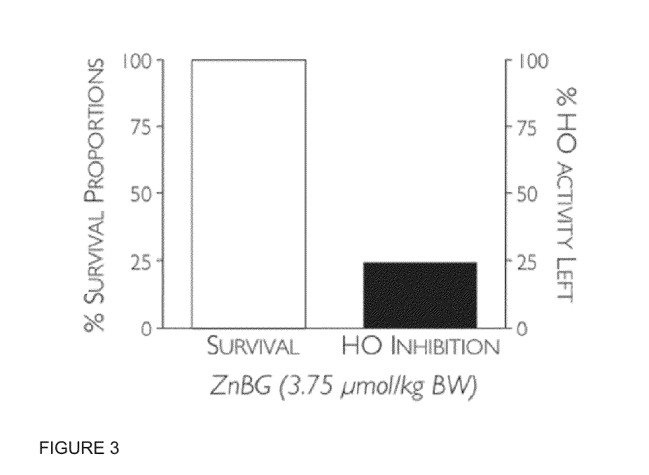

There was no significant difference in survival between either group. In contrast to CrMP, pups given 30 μmol/kg ZnBG and kept in the dark did not show any chemical toxicity. However, after exposure to light, we found an LD₅₀ of 19.5 μmol/kg and that pups had significant weight loss, decreases in liver antioxidant capacities, and increases in creatine kinase (CK-MB) and aspartate aminotransaminase (AST) levels. In addition, 6 days after light exposure, ZnBG-treated pups developed gross histologic skin changes at doses >7.5 μmol/kg BW. Interestingly, no lethality was observed in pups treated with 30-μmol ZnBG/kg BW and exposed to blue light-emitting diode (LED) light. In summary, we showed that CrMP has a chemical toxicity, but no phototoxicity. It appeared that ZnBG has no chemical toxicity, but a phototoxicity that is dependent on the light source. Most importantly, low doses of ZnBG (<3.75 μmol/kg BW) retained maximal HO inhibitory potency without photosensitizing effects (FIG. 3).

Decision to use zinc protoporphyrin (ZnPP). We have focused our studies on naturally occurring ZnPP, another favorable compound with a balance between positive and negative characteristics. It is a Mp with sufficient potency (dose that inhibits enzyme activity by 50%, i.e. IC₅₀) spleen and brain: approximately 6.0 and 3.0 μM, respectively, in the rat and mouse); does not measurably cross the blood brain barrier; is not photochemically active; minimally upregulates HO-1 in cell culture or in newborn mice; has a rapid onset of action; short-acting; and is not degraded by HO. Also, this compound could be administered in a low dose that primarily affects HO-1, but not nitric oxide synthase (NOS) or basal soluble guanylyl cyclase (sGC) activity to any significant extent.

However, ZnPP has been troublesome to maintain in solution and is not orally absorbed, requiring parenteral administration. In a model of hemolytic jaundice where newborn rhesus monkeys are treated with heat-damaged erythrocytes, we found that ZnPP at a dose of 40 μmol/kg BW given subcutaneously (SC) effectively reduced carboxyhemoglobin and total bilirubin levels to baseline levels within 24 hours. HO inhibition was targeted to the liver and spleen, without affecting the kidney or brain.

Thus, this compound is very promising for use in the treatment of neonatal jaundice. Although not effective after oral administration by itself, formulations using polymeric particulate delivery systems allow oral bioavailability and enhance gastric passage. We have previously shown that incorporating Mps into liposomes may significantly increase delivery to the spleen and thus enhanced their efficacy. The need for oral delivery is important as not all infants have intravenous (IV) access for parenteral administration of drugs, such as late preterm and term infants being readmitted for hyperbilirubinemia. Moreover, oral administration results in targeted delivery of HO inhibitors, taking advantage of the “first pass effect” to the liver and spleen, the target organs; whereas IV administration results in systemic distribution. Thus, we have designed and developed formulations using drug delivery systems that increase oral bioavailability and gastric passage to result in targeted delivery of Mps to the liver and spleen. Such polymeric particulate drug delivery systems (micro- or nanoparticles) improve drug stability and enhance gastric passage for use in the human neonate.

Using this approach, we evaluated a number of formulations that have successfully improved the oral bioavailability and effectiveness of ZnPP. Use of polymeric particulate delivery systems (micro- and nanoparticles) provides delivery approaches that can both improve stability against degradation and enhance intestinal absorption. In our studies, ZnPP/lipid microparticles were prepared by controlled cation-induced micro-emulsion or spray-drying. In particular, DPSS (1,2-dipalmitoyl-sn-glycero-3-phosphocholine) and DPSC (distearoyl-sn-glycerophosphocholine) are biodegradable phospholipids approved by the FDA for use in premature infants as surfactants.

The Zinc Formulations. We designed five different formulations of ZnPP using polymeric particulate delivery systems (micro- or nanoparticles) to improve its stability and enhance its intestinal absorption. Enterically-coated microparticles were designed to not only protect ZnPP from the acidic environment of the stomach, which we have found to render ZnPP inactive, but also to maximize its release in pH>5.5 during transit to the small intestine. The methacrylic co-polymer (Eudragit® L100-55, insoluble at pH<5.5) was used in combination with DPPC and/or DSPC, or phospholipids only. These phospholipids are FDA-approved excipients, and are endogenous phospholipids, used as the main constituents of artificial lung surfactant previously approved for use in premature newborns in high concentrations. The microparticles are formed by emulsion or spray-drying techniques.

The ZnPP formulations that were prepared and tested are shown in Table 1. The formulations made using emulsions were stored frozen and lyophilized to obtain the final chitosan-based microparticles (ZnPP-A and ZnPP-B). The formulations made using spray-drying were stored as dry powders and frozen. Although alginate and chitosan are biodegradable polymers approved by the FDA for adult human use, they are not approved for use in premature infants. Further consideration of emulsions approved for human use led us to the synthesis of the ZnPP-Poly using acrylic beads (e.g., EUDRAGIT®). In addition, a ZnPP Lipid preparation was created consisting of FDA-approved phospholipids, DPPC and DSPC.

TABLE 1

| Emulsion | Spray-drying |
|---|---|
| Chitosan-based: ZnPP-A | EUDRAGIT ®-based: ZnPP-poly |
| ZnPP-B | Lipid-based: ZnPP-lipid |

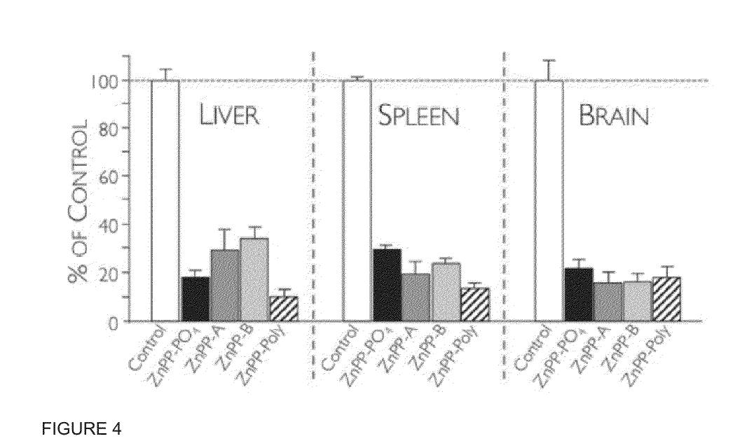

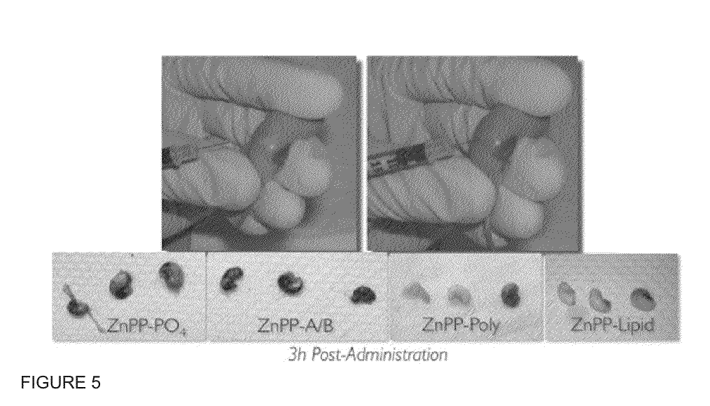

Evaluation of the ZnPP Formulations. We tested the in vitro HO inhibitory potency of the ZnPP preparations (ZnPP-A, ZnPP-B, and ZnPP-Poly) against our aqueous sodium phosphate prepared in 0.4 M Na₃PO₄, normal saline, and distilled water (ZnPP-PO₄) in tissue sonicates of liver, spleen, and brain harvested from 3-day-old mouse pups. We found that the ZnPP-Poly was the most potent towards inhibiting liver HO activity followed by our ZnPP-PO₄ solution (FIG. 4). The chitosan/alginate-based formulations had the least potency. All formulations had similar potency in spleen and brain tissues. Based on these promising findings and the design of the ZnPP-Lipid, we then evaluated the in vivo HO inhibitory potency of these ZnPP formulations compared to the ZnPP-PO₄ solution. 3-day-old mice were given vehicle (VEH) or 30 μmol/kg BW of one of the five ZnPP formulations or VEH alone by direct intragastric (IG) injections (FIG. 5). After 3 h, pups were sacrificed and livers, spleens, and brains were harvested for measurements of HO inhibitory potency. HO activity was calculated as pmol CO/h/mg fresh weight (FW) and then expressed as mean±SD% of control values.

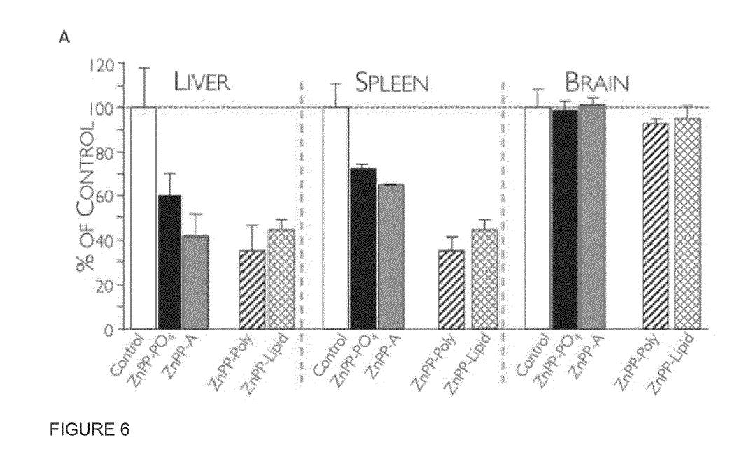

To evaluate toxicity, pups were treated similarly and then immediately placed for 3 hours under fluorescent tubes (2 cool white/1 blue TL52) to test for phototoxicity or kept in the dark to test for chemical toxicity. Overall survival of pups was then monitored up to 1 week. Even though ZnPP-PO₄ was found to be the most potent (80%) in vitro (FIG. 4), its potency significantly decreased after IG administration (FIG. 6A), probably due to its poor gastric passage (FIG. 5). As expected, it showed no phototoxicity or chemical toxicity (FIG. 6B).

The ZnPP chitosan/alginate-based preparations were potent in vivo, but because chitosan/alginate is not FDA-approved for use in premature infants, these formulations were not evaluated further for toxicity. The ZnPP-Poly was the most potent in liver and spleen (FIG. 6A), but it was phototoxic, resulting in a mortality of 90% 48 hours after light exposure (FIG. 6B), which may have been due to the polymer itself as Poly-Only-treated pups had a 100% and 70% mortality after light and dark exposures, respectively. Importantly, we observed that the ZnPP-Lipid formulation was also potent, but showed no photo- or chemical toxicity. Because the ZnPP-Lipid formulation was effective in inhibiting liver HO activity after IG administration and had no toxicity, we thus concluded that it has the most potential for use in the treatment of neonatal jaundice.

These data show that the naturally-occurring ZnPP is potent and has rapid action without long-term inhibitory effects, making it highly useful for an anti-hyperbilirubinemia drug intended for use in treating newborns during the transitional period. Designing ZnPP to be orally bioavailable while maintaining these efficacy and safety characteristics gives pediatricians confidence in its use for the control of bilirubin levels in targeted newborn infants with hemolysis or other causes of increased bilirubin production.

### EXAMPLE 2

### Formulations

One disadvantage of ZnPP is that it is not orally bioavailable and it needs to be administered parenterally. Low oral bioavailability of ZnPP is a consequence of its low solubility and chemical instability in low pH environments, like that found in the stomach, and low aqueous solubility at neutral pH that limits its dissolution and subsequent absorption in the intestinal tract. ZnPP reacts in low pH aqueous solutions to form protoporphyrin IX free acid, which is inactive to inhibit HO.

A formulation is needed that will improve the oral bioavailability and effectiveness of ZnPP by both protecting the molecule from interacting with the acid environment found in the stomach and increasing its aqueous solubility in neutral pH to promote absorption in the upper small intestine. Ideally the formulation is in solid state in the form of a powder to improve both shelf-life of the eventual pharmaceutical dosage form as well as its manufacturability.

Microparticles are formed by spray drying which ensures a straightforward path for scale-up under a GMP environment which will be required for the GMP manufacturing of a final dosage form.

Spray dried powder preparation; formulation components:

ZnPP; 20% w/w

DPPC; 5% w/w

EUDRAGIT® L100-55; 75% w/w

Preparation of 1% solids (w/v) feedstock solution for spray drying: ZnPP is dissolved with sonication in 1 M ammonium hydroxide (constituting 12.5% of total solvent volume). DPPC is added dissolved in ethanol (12.5% of total solvent volume). EUDRAGIT® L100-55 is added dissolved in ethanol (remaining 75% solvent volume).

Büchi-290 spray drying conditions:

Inlet temperature: 70° C.

Outlet temperature: 50° C.

Aspirator: 75%

Nitrogen: 30 mm gauge height

Pump: 8%

The feeding solution container and spray dryer compartments are protected from light during the process. The dry powder is stored frozen protected from light.

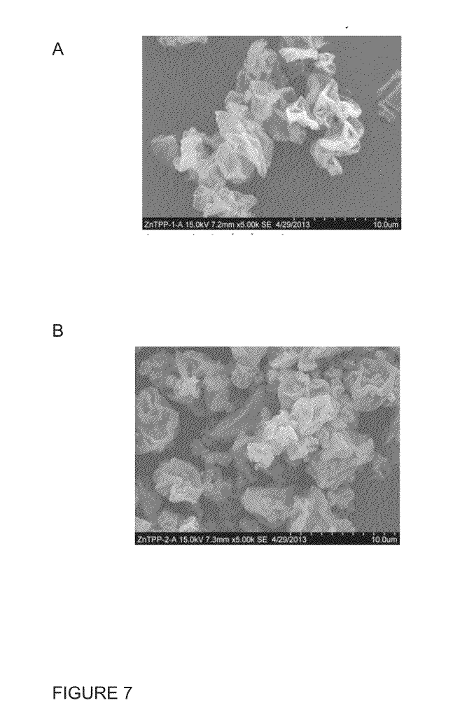

Observations on this formulation, shown in FIG. 7A: The 20% ZnPP content was verified by LCMS. Limited ZnPP release is evident in 0.1 N HCl medium, washing and resuspension of the acid-exposed particles in PBS pH 7.4 buffer resulted in large release of ZnPP measured by HPLC/MS. SEM images for spray dried particles containing 75% w/w EUDRAGIT®. Wrinkled morphology indicates early polymer precipitation during particle formation ensuring efficient drug encapsulation.

### EXAMPLE 3

### Spray Dried Powder Preparation With Higher ZnPP Content

ZnPP formulation with lower EUDRAGIT® content; formulation components:

ZnPP; 20% w/w

DPPC; 42% w/w

EUDRAGIT® L100-55; 38% w/w

Preparation of 1% solids (w/v) feedstock solution for spray drying: ZnPP is dissolved with sonication in 1 M ammonium hydroxide (constituting 12.5% of total solvent volume). DPPC is added dissolved in ethanol (50% of total solvent volume). EUDRAGIT® L100-55 is added dissolved in ethanol (remaining 37.5% solvent volume). Spray drying conditions: same as described above.

Observations on this formulation, shown in FIG. 7B: The 20% ZnPP content was verified by LCMS. Even though some ZnPP release is evident in 0.1 N HCl medium, washing and resuspension of the acid-exposed particles in PBS pH 7.4 buffer resulted in large release of ZnPP measured by HPLC/MS. SEM images show similar morphology for these 38% w/w EUDRAGIT® particles compared to the previously tested formulation (75% w/w EUDRAGIT®). Wrinkled morphology indicates early polymer precipitation during particle formation ensuring efficient drug encapsulation.

### EXAMPLE 4

### Powder Containing Vial or Syringe to be Reconstituted With an Appropriate Diluent to be Orally Delivered to an Infant Through a Feeding Tube

The desired dose of ZnPP may be contained in a small amount of spray-dried powder or less depending on the final ZnPP content and depending the target subject (e.g. newborn mouse or rat, monkey, or an infant patient through a feeding tube). Spray-dried powders are usually small in size and tend to be more cohesive than granular powders. A bulking agent can be used to blend the spray-dried powder to facilitate filling into a vial to be then resuspended with an appropriate diluent prior to administration to the test subject (newborn mouse or rat, monkey, etc.) or to an infant patient through a feeding tube. This can be achieved as described below.

ZnPP-DPPC-EUDRAGIT® spray-dried powder is mixed with D-glucose as bulking agent to obtain a uniform mixture containing the target amount of spray dried powder, calculated based on the amount of ZnPP content and the required dose, with an appropriate amount of D-glucose (ranging in the amount of 10% to 90% w/w) the powder blend can then be filled in a glass or plastic vial or syringe manually or using a filling machine.

Prior to administration, a suspension is then formed by adding the appropriate amount of diluent containing 0.25% (w/v) citrate buffer, pH 4.7, to the target amount of powder containing the required dose. The pH of the diluent is key to minimize dissolution of the polymer microparticles, but not too low to cause chemical degradation of ZnPP. The suspension is agitated and ready for administration.

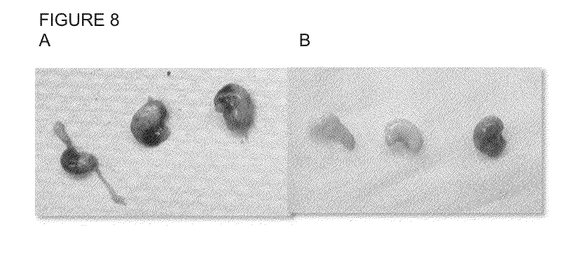

FIG. 8A, ZnPP-PO₄ administered in a sodium phosphate solution showing precipitation of protoporphyrin. FIG. 8B. 3-hour post-administration of ZnPP spray-dried microparticles. Precipitation is completely inhibited in two of the three 3-day-old mice pups treated.

### EXAMPLE 5

### ZnPP Oral Thin Film Dosage Form

The ZnPP spray-dried powder formulation can be dispersed in a solution of an organic solvent containing a film-forming polymer. The polymer solution containing the suspended spray-dried powder is then casted into a thin film, which is then cut in appropriate size sections, each of the sections containing one dose. The thin film is expected to instantaneously dissolve in the infant patient mouth without the need of any extra liquid.

### EXAMPLE 6

### ZnPP Fast Dissolve Tablet

The ZnPP spray-dried powder formulation can be suspended in diluent containing 0.25% (w/v) citrate buffer, pH 4.7 to which mannitol can be added in an appropriate amount. The suspension is then transferred into tablet-size molds, which then are frozen in a stream of liquid nitrogen. The frozen suspension is then lyophilized and the mold containing the tablets obtained can be sealed with a protective cover. The lyophilized tablets are expected to instantaneously dissolve in the infant patient mouth without the need of any extra liquid.

### EXAMPLE 7

### Preparation of ZnPP/Chitosan Microparticles

Formulation contents (w/w): Coconut oil (40%), Lecithin (30%), ZnPP (5%), Poloxamer-188 (20%) and Chitosan (MW-15000) (5%)

Preparation procedure: Coconut oil (40 mg) and Lecithin (30 mg) were dissolved in 10 mL of ethanol by stirring at room temperature (RT). To this solution, ZnPP (5 mg) was added and heated to 40° C. while stirring to obtain a clear solution. Separately, Poloxamer-188 (20 mg) was dissolved in 8 mL of distilled water and then added to the above ethanol solution while stirring at room temperature. Chitosan-15K (5 mg) was dissolved in 1 mL of 0.01 N aqueous hydrochloric acid and then added to the above mixture solution stirring at room temperature. The solvents were removed from this homogeneous solution under vacuum using a rotary evaporator. The residue was reconstituted with 10 mL of water by sonication for 20 min. The solution was frozen and lyophilized to obtain the final Zn-PP/chitosan microparticles.

### EXAMPLE 8

### Preparation of ZnPP/Alginate Microparticles

Formulation contents (w/w): Coconut oil (35%), Lecithin (20%), ZnPP (20%), Poloxamer-188 (20%) and Sodium Alginate (4.5%) Calcium chloride (0.5%)

Preparation procedure: Coconut oil (35 mg) and Lecithin (20 mg) were dissolved in 10 mL of ethanol by stirring at room temperature. To this solution, ZnPP (20 mg) was added and heated to 40° C. while stirring to obtain a clear solution. Separately, Poloxamer-188 (20 mg) and Sodium Alginate (4.5 mg) were dissolved in 8 mL of distilled water and then added to the above ethanol solution while stirring at room temperature. Calcium chloride (0.5 mg) was dissolved in 1 mL of water and then added to the above mixture solution stirring at room temperature. The solvents were removed from this homogeneous solution under vacuum using a rotary evaporator. The residue was reconstituted with 10 mL of water by sonication for 20 min. The solution was frozen and lyophilized to obtain the final ZnPP/alginate microparticles.

### EXAMPLE 9

### Preparation of ZnPP/Sodium Trimetaphosphate (STMP) Microparticles

Formulation contents (w/w): Coconut oil (35%), Lecithin (20%), ZnPP (20%), Poloxamer-188 (20%) and Sodium trimetaphosphate (STMP) (5%)

Preparation procedure: Coconut oil (35 mg) and Lecithin (20 mg) were dissolved in 10 mL of ethanol by stirring at room temperature. To this solution, ZnPP (20 mg) was added and heated to 40° C. while stirring to obtain a clear solution. Separately, Poloxamer-188 (20 mg) and STMP (5 mg) were dissolved in 8 mL of distilled water and then added to the above ethanol solution while stirring at room temperature. The solvents were removed from this homogeneous solution under vacuum using a rotary evaporator. The residue was reconstituted with 10 mL of water by sonication for 20 min. The solution was frozen and lyophilized to obtain the final ZnPP/STMP microparticles.

### EXAMPLE 10

### Preparation of ZnPP Microparticle Formulations

Formulation contents (w/w): DSPC (45%), DPPC (45%), ZnPP (10%).

Preparation procedure: ZnPP (10 mg) was dissolved with sonication in 10 mL of 1 M ammonium hydroxide solution. DPPC (45 mg) dissolved in 45 mL ethanol was added to it. DSPC (45 mg) dissolved in 45 mL of ethanol was added and mixed well. The solution mixture was spray dried using Buchi-290 mini spray dryer. The set parameters were inlet temperature 55° C., outlet temperature 50° C., aspirator at 75%, solution feed pump at 8% and nitrogen flow at 30 mm. The feeding solution container and spray dryer compartments are protected from light during the process. The dry powder is stored frozen protected from light.

### EXAMPLE 11

### In Vivo Inhibition of Heme Oxygenase Activity in the Heme-Loaded Newborn Mouse Using a Microparticle Formulation of Zinc Protoporphyrin

Heme oxygenase (HO), the rate-limiting enzyme in heme degradation, produces bilirubin. Because hemolysis can lead to increased bilirubin production and cause neonatal hyperbilirubinemia, inhibition of HO, e.g. by metalloporphyrins (Mps), may be an ideal strategy. Zinc protoporphyrin (ZnPP) is a promising Mp as it is naturally occurring, potent, not phototoxic, and has minimal HO-1 upregulation, but its use has been limited since it is not orally absorbed.

We designed a lipid-based formulation of ZnPP (ZL) as described in Example 10 and have shown that it is effective and safe in inhibiting liver HO activity after oral administration in a newborn mouse model. We further extend these studies to investigate the efficacy and safety of ZL in the heme-loaded newborn mouse, a model analogous to that of a hemolyzing infant. The ZL was prepared as described in Example 10, and resuspended in PBS at the concentrations indicated.

3d-old FVB pups were given 30 pmol/kg of heme (H) or vehicle (V) by SQ injections. 24 h post-heme treatment, mice were given V (H-V) or ZL (1.8-60 μmol/kg, H-ZL1.8-H-ZL60) via intragastric injections. 3 h later, pups were sacrificed and livers and brains harvested for measurements of HO activity by gas chromatography. Upregulation of HO-1 was assessed by determinations of liver HO-1 mRNA and protein levels by RT-PCR and Western Blots, respectively. Data were expressed as % of controls.

After a heme load (H-V), liver HO activity significantly increased 1.6-fold as expected (Table 2). This heme-induced increase in HO activity was inhibited in a dose-dependent manner after treatment with ZL, with HO activity returning to control levels at a dose of 30 μmol/kg. No significant inhibition of brain HO activity or changes in liver HO-1 mRNA and protein levels were found after administration of 30 μmol ZL/kg.

ZL at a dose of 30 μmol/kg effectively inhibits liver HO activity after heme loading. In addition, it does not appear to cross the blood/brain barrier or induce HO-1 mRNA or protein levels. We conclude that ZL is effective and safe and thus is an attractive compound for use in treating neonatal hyperbilirubinemia due to hemolysis.

TABLE 2
HO Activity (% of Control, Mean ± SD)

| Group | V-V | H-V | H-ZL1.8 | H-ZL3.8 | H-ZL7.5 | H-ZL15 | H-ZL30 | H-ZL60 |
|---|---|---|---|---|---|---|---|---|
| Liver | 100 ± 14* | 164 ± 28† | 138 ± 34†* | 122 ± 11†* | 118 ± 18†* | 115 ± 22†* | 105 ± 19* | 91 ± 15* |

p < 0.05 vs †V-V or *H-V,
n = 7-35 per group

### EQUIVALENTSquivalents

Those skilled in the art will recognize, or be able to ascertain using no more than routine experimentation, many equivalents to the specific embodiments and methods described herein. Such equivalents are intended to be encompassed by the scope of the present invention.

All patents, patent applications, and literature references cited herein are hereby expressly incorporated by reference.

What is claimed is:

1. A microparticle comprising:
ZnPP and a pharmaceutically acceptable stabilizer, where the concentration of ZnPP is from about 5% by weight of the microparticle.

2. The microparticle of claim 1, wherein the concentration of ZnPP in the microparticle is from about 5% to about 50% by weight of the microparticle.

3. The microparticle of claim 1, wherein the microparticle has increased stability in acidic conditions as compared to ZnPP alone.

4. The microparticle of claim 3, wherein the microparticle is at least 10% more stable to acidic conditions than ZnPP alone.

5. The microparticle of claim 3, wherein the microparticle is two-fold more stable to acidic conditions than ZnPP alone.

6. The microparticle of claim 1, wherein the microparticle has enhanced solubility at neutral pH as compared to ZnPP alone.

7. The microparticle of claim 6, wherein the microparticle is at least 10% more soluble at neutral pH than ZnPP alone.

8. The microparticle of claim 6, wherein the microparticle is at least 2-fold more soluble at neutral pH than ZnPP alone.

9. The microparticle of claim 1, wherein the microparticle consists essentially of ZnPP and the pharmaceutically acceptable stabilizer.

10. The microparticle of claim 1, wherein the microparticle has a diameter of from about 10 nm to about 10 μm.

11. microparticle claim 1, wherein the microparticle has a diameter of from about 100 nm to about 5 μm.

12. microparticle of claim 1, wherein the stabilizer is alginate, chitosan, lecithin, a poloxamer, sodium trimetaphosphate, a cationic lipid, a protein, an oil, polyvinylpyrrolidone, or a combination thereof.

13. The microparticle of claim 1, wherein the stabilizer is a cationic lipid or a mixture of cationic lipids.

14. The microparticle of claim 13, wherein the cationic lipid is DSPC, DPPC, DOTIM, DDAB, DOTMA, DMRIE, EDMPC, DCChoI, DOGS, MBOP, or any combination thereof.

15. microparticle of claim 1, wherein the concentration of ZnPP is from about 10% to about 25% by weight of the microparticle, and the stabilizer is a mixture of DSPC and DPPC.

16. The microparticle of claim 15, wherein the weight ratio of DSPC to DPPC is about 1:1.

17. microparticle of claim 1, wherein the stabilizer comprises (i) lecithin, (ii) a poloxamer, and (Hi) alginate, sodium trimetaphosphate, or chitosan.

18. The microparticle of claim 17, wherein the concentration of ZnPP is from about 5% to about 25% by weight of the microparticle.

19. The microparticle of claim 17, wherein the concentration of lecithin is from about 10% to about 40% by weight of the microparticle, and the concentration of the poloxamer is from about 10% to about 40% by weight of the microparticle.

20. The microparticle of claim 17, wherein the stabilizer further comprises an oil.

21. The microparticle of claim 17, wherein the stabilizer comprises sodium trimetaphosphate.

22. The microparticle of claim 21, wherein the concentration of sodium trimetaphosphate is from about 3% to about 6% by weight of the microparticle.

23. The microparticle of claim 17, wherein the stabilizer comprises alginate.

24. The microparticle of claim 22, wherein the concentration o alginate is from about 3% to about 6% by weight of the microparticle.

25. The microparticle of claim 17, wherein the stabilizer comprises chitosan.

26. The microparticle of claim 25, wherein the concentration of chitosan is from about 3% to about 6% by weight of the microparticle.

27. A composition comprising a plurality of microparticles of claim 1 and, optionally, a pharmaceutically acceptable carrier.

28. A composition of claim 27, in a unit dose for oral administration.

29. composition of claim 27, wherein the plurality of microparticles are suspended in the pharmaceutically acceptable carrier.

30. A method of inhibiting the activity of inducible heme oxygenase (HO-1) in a subject in need thereof, the method comprising:
administering to the subject an effective amount of composition according to claim 27.

31. The method of claim 30, wherein the administering is oral.

32. The method of claim 30, wherein the subject is a human infant with hyperbilirubinemia.

33. The method of claim 30, whereby the activity of inducible HO-1 is inhibited as compared to the activity of inducible HO-1 in a control subject, or the activity of inducible HO-1 is inhibited as compared to the activity of inducible HO-1 prior to administration of the microparticles.
